(19) United States
(12) Patent Application Publication
(10) Pub. No.: US 2008/0050805 A1
(43) Pub. Date: Feb. 28, 2008

(54) Tissue board assembly

(76) Inventors: John P. Cole, Alpharetta, GA (US); Tesfaye H. Gutema, Alpharetta, GA (US)

Correspondence Address: BERKELEY LAW & TECHNOLOGY GROUP, LLP, 17933 NW Evergreen Parkway, Suite 250, BEAVERTON, OR, 97006, US

(21) Appl. No.: 11/508,387

(22) Filed: Aug. 22, 2006

(51) Int. Cl. C12M 3/00

(52) U.S. Cl. 435/289.1

(57) ABSTRACT

A Tissue Board Assembly for collecting, storing, preparing and preserving tissue and/or bone.

### BACKGROUND

This disclosure relates to a surface for collection of tissue and bone samples taken during a surgical procedure. Surfaces for collecting tissue grafts and samples are used during surgical procedures.

During various surgical procedures, collection, preservation and treatment of tissue specimens collected during the procedure may be accomplished. One such procedure is a follicular dissection and transplantation. During a follicular dissection, the collection, preservation and treatment of hair follicle grafts harvested is implemented in order to ensure successful transplantation of hair follicles. Follicular dissection is accomplished by gently isolating hair follicles and skillfully extracting them from a donor site with the help of forceps and special graft extracting devices. The extracted grafts may then be transferred to sterile saline solution with a temperature between 0-8° C. After grafts are collected they may require further processing such as stripping, measuring, stretching, fat removal and dissection into follicles with a smaller number of hairs. Another surgical procedure requiring processing of harvested tissue is strip hair transplantation which is strip harvesting of skin tissue having hair follicles from one area of the body for transplantation to another area of the body. Here a surgeon completes the excision in the donor area and an assistant receives the strip in a sterile cup of saline solution having a temperature greater than 0C and less than or equal to about 8° C. The strip may then be processed by slivering the strip into smaller sections. Each sliver is dissected out with a scalpel, freeing it (slivering) from the rest of the tissue. The collected slivers may be stored in a shallow dish of saline with a temperature greater than 0° C. and less than or equal to about 8° C. The slivers are then dissected into grafts ready to be placed in the recipient area. The method of collection, treatment and preservation of hair follicles and/or slivers collected during a follicular dissection and transplantation procedure may have a direct effect on the yield of successfully transplanted follicles or slivers to the donor site.

### BRIEF DESCRIPTION OF THE DRAWINGS

FIG. 1 is a diagram of a tissue board assembly according to a particular embodiment.

FIG. 2 is a diagram of a tissue board assembly circuit and pump according to a particular embodiment.

FIG. 3 is a diagram of a tissue board assembly comprising a cavity according to a particular embodiment.

FIG. 4 is a diagram of a tissue board assembly comprising a cavity according to a particular embodiment.

FIG. 5 is a diagram of a tissue board assembly comprising an interior region for circulating a substance according to a particular embodiment.

FIG. 6 is a diagram of a tissue board assembly comprising a cavity according to a particular embodiment.

FIG. 7a is a diagram of a tissue board assembly according to a particular embodiment.

FIG. 7b is a diagram of a tissue board assembly according to a particular embodiment.

FIG. 8 is a diagram of a tissue board assembly according to a particular embodiment.

FIG. 9 is a diagram of a tissue board assembly comprising a removable insert according to a particular embodiment.

FIG. 10a is a diagram of a removable container with connectors according to a particular embodiment.

FIG. 10b is a diagram of a removable container with connectors according to a particular embodiment.

FIG. 10c is a diagram of a removable container with connectors according to a particular embodiment.

FIG. 11 is a diagram of a Disposable Tissue Board assembly as a cover for a TBA according to a particular embodiment.

FIG. 12 is a diagram of a Disposable Tissue Board as a stand-alone device according to an embodiment.

### DETAILED DESCRIPTION

In the following detailed description, numerous specific details are set forth in order to provide a thorough understanding of claimed subject matter. However, it will be understood by those skilled in the art that claimed subject matter may be practiced without these specific details. In other instances, well-known methods, procedures, and components have not been described in detail so as not to obscure claimed subject matter.

Although the examples and embodiments described herein refer to tissue samples taken during a follicular dissection procedure or a “strip hair transplantation”, such examples are meant for illustrative purposes and are not intended to limit the scope of the disclosure. The disclosed device and method is useful for collection, preservation and treatment of any of a variety of tissue and/or bone specimens collected during any of a variety of surgical procedures. Such tissues may include skin, collagen, muscle tissue, ligament, tendon, vascular, neural and/or bone graft. Additionally, such surgical procedures may include biopsy, cosmetic plastic surgery, reconstructive burn surgery and ACL surgery, for instance.

A Tissue Board Assembly (TBA) is discussed below. A TBA may comprise a variety of materials such as polymeric materials such as thermoplastics, thermosets, and/or elastomers, and/or metals such as, for instance, stainless steel, titanium and/or aluminum. The material may be transparent to allow light through, resistant to wear, Food and Drug Administration (FDA) compliant. However, these are merely examples of types of materials that may be used to make a TBA and claimed subject matter is not limited in this regard. A TBA may be obtained by machining, molding and/or vulcanizing processes. Various features of a TBA may comprise different materials. For instance, a main board may comprise plastic materials like polycarbonate and inserts, connectors and fasteners may be made of other type materials like metal, glass, and/or ceramic. However, these are merely examples of various materials comprising and methods of making a TBA and claimed subject matter is not limited in this regard.

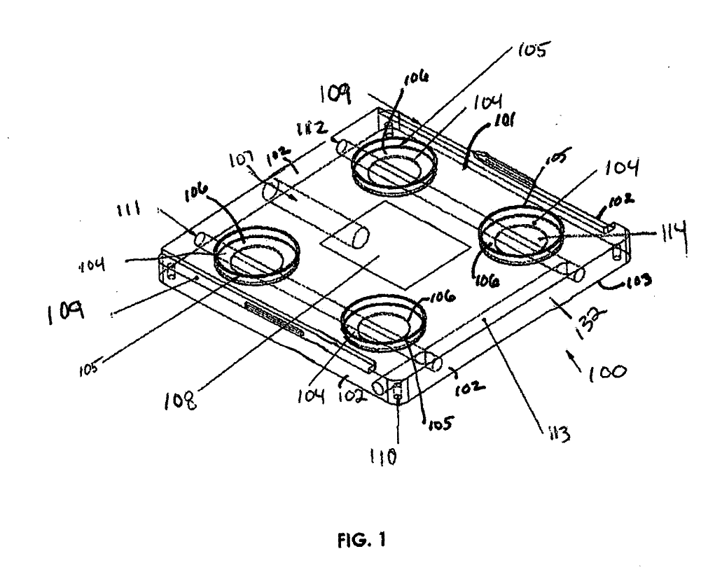

FIG. 1 illustrates a particular embodiment of a Tissue Board Assembly (TBA) comprising at least one fluid circulation member. In a particular embodiment a TBA 100 may be used during a follicular dissection procedure and may be characterized by a top surface 101, lateral surfaces 102 and a bottom surface 103. In a particular embodiment a TBA 100 may have a three dimensional shape. According to a particular embodiment, a TBA 100 may have dimensions such as 6 inches in length, 6 inches in width and 0.5 to 1 inch in height. However, this is merely an example of dimensions that a TBA 100 may have and claimed subject matter is not limited in this regard.

In a particular embodiment, a TBA 100 may have insert pockets 104. Insert pockets 104 may have a variety of shapes, such as for instance round and/or rectangular. According to a particular embodiment, a TBA 100 may have four insert pockets 104 each having substantially the same dimensions. However, this is merely an example of the number of insert pockets 104 a TBA may have and claimed subject matter is not so limited. Insert pockets 104 may have a depth of about 0.25 inches from the top surface 101 of TBA 100. Insert pockets 104 may have a bottom inner surface 106 lying substantially in a plane parallel to top surface 101. Bottom inner surface may have a variety of shapes, such as, for instance, flat, concave, convex and/or stepped. Additionally, insert pockets 104 may have a water tight joint (not shown) at the interface between the insert and the main board for assembly with containers 105. A water tight joint may be provided during assembly, by fitting an O-ring (not shown) between lateral walls of insert pocket 104 and container 105. Such an O-ring may be made of an elastomeric material and may be compressed between insert pocket 104 and container 105 providing a seal. However, these are merely examples of dimensions and features of insert pockets and claimed subject matter is not limited in this regard.

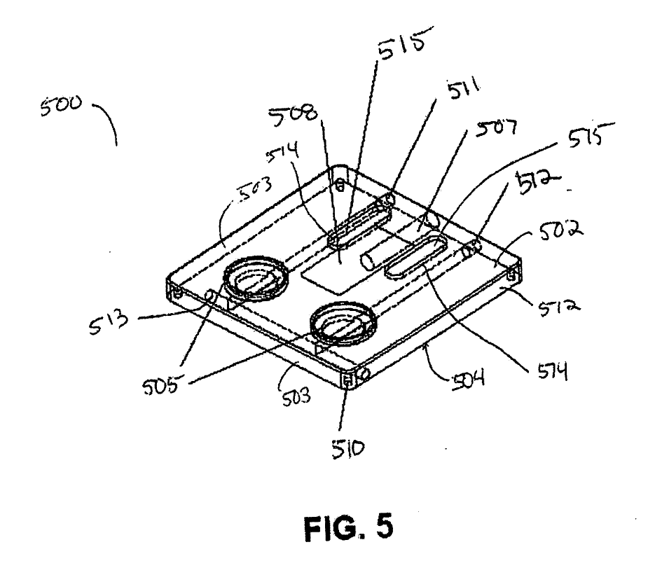

Referring still to FIG. 1, in a particular embodiment, containers 105 may be disposed within insert pockets 104. During a follicular dissection procedure, containers 105 may be useful for preserving tissue grafts removed during the procedure. Although four containers 105 are shown in FIG. 5, TBA may include more or fewer containers 105 and associated structures, and the scope of the claimed subject matter is not limited in this respect. Such containers 105 may contain a liquid such as saline, balanced salt solution, and/or an appropriate solution for preserving tissue grafts. According to a particular embodiment, containers 105 may be made of a variety of materials such as, for instance, materials having a higher coefficient of thermal conductivity. Such materials may include stainless steel, titanium, and/or glass, for instance. However, these are merely examples of materials that containers may be made from and claimed subject matter is not limited in this regard.

In a particular embodiment, TBA 100 may comprise at least one light source, such as, for instance a fiber optic light source (not shown). A light source may be accessed, for instance, via a light access region 107. According to a particular embodiment, light access region 107 may be a variety of diameters. Additionally, light access region 107 may be a blind bore hole or may be a through hole. Alternatively, an external light source (not shown) may also be used. However, these are merely examples of positions and sizes for a light access region and claimed subject matter is not limited in this regard.

In a particular embodiment, a work area 108 may be disposed on top surface 101. According to a particular embodiment, work area 108 may have a variety of shapes and have a variety of surface areas. Work area 108 may be textured, for example. Such texturing of work area 108 may prevent tissue from slipping and may assist a user while preparing tissue during a follicular dissection procedure. However, this is merely an example of a work area that may be disposed on a top surface of a TBA and claimed subject matter is not limited in this regard.

In a particular embodiment, a TBA 100 may comprise one or more tool rests 109 which may be used to store the working tools, such as, for instance, scalpel blades and forceps. Tool rests 109 may be disposed in a variety of locations on a TBA 100, such as, for instance on lateral sides of top surface 101. According to a particular embodiment, tool rests 109 may be recesses or pockets. However, these are merely examples of tool rest positions and styles and claimed subject matter is not limited in this regard.

In a particular embodiment, TBA 100 may comprise at least one foot 110 (feet 110) disposed on bottom surface 103. Feet 110 may be assembled onto TBA 100 by threading, gluing, and/or force fitting. According to a particular embodiment, feet 110 may be made of a variety of substances, such as, for instance, rubber, plastic and/or metal. Feet 110 may help prevent TBA 100 from slipping during a follicular dissection procedure. However, this is merely an example of feet for a TBA and claimed subject matter is not limited in this regard.

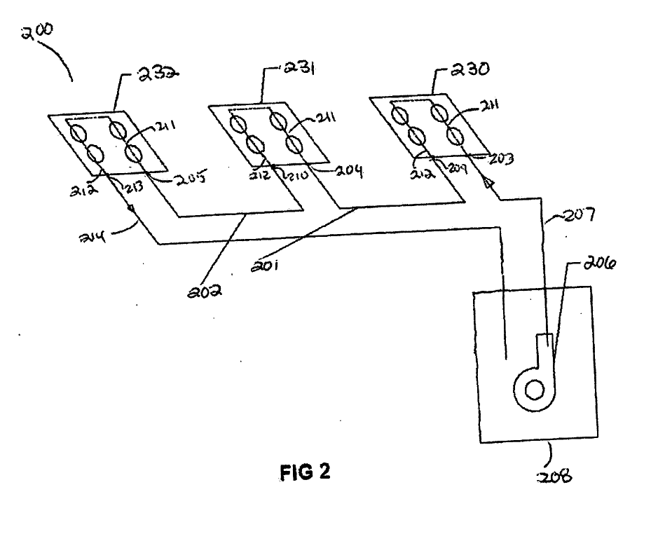

Referring still to FIG. 1, in a particular embodiment, TBA 100 may comprise inlet 111, outlet 112 and feeder line 113. Inlet 111, outlet 112 and feeder line 113 may comprised interior regions bored through the thickness of main board 132. Feeder line 113 may comprise a blind hole going through the thickness of main board 132. Feeder line 113 may be coupled to inlet 111 and outlet 112 within the thickness of the main board 132. Inlet 111 may open on a lateral side of main board 132 and terminate at feeder line 113 and outlet 112 may also open on a lateral side of main board 132 and terminate at feeder line 113. However, this is merely an example of a configuration of inlets, outlets and feeder lines and claimed subject matter is not limited in this regard. According to a particular embodiment a fluid and/or gas may flow through inlet 111, outlet 112 and feeder line 113. Such fluid and/or gas may be, for instance, oil, water, water-glycol, air and/or liquid nitrogen. However these are merely examples of various fluids and/or gases that may flow through inlets, outlets and feeder lines and claimed subject matter is not limited in this regard. Such fluid and/or gas may also flow into and out of at least one reservoir 114 via inlet 111, outlet 112 and feeder line 113. In a particular embodiment, a reservoir 114 may be disposed adjacent to an insert pocket 104. According to a particular embodiment, reservoir 114 may be in thermal contact with insert pocket 104. Such thermal contact may allow heat to be transferred between insert pocket 104 and fluid and/or gas flowing through inlet 111 and/or outlet 112. According to a particular embodiment, reservoirs 114 may have a variety of shapes and dimensions, such as, for instance, reservoir 114 may be a substantially circular cavity having a depth of about 0.25 inch and a diameter of about 1.5 inches. Fluid and/or gas flowing through inlet 111, outlet 112 and feeder line 113 may warm or cool substances contained in containers 105. Fluid and/or gas may be cooled or warmed to a desired temperature before entering inlet 111 as described with reference to FIG. 2. Such a desired temperature may be between 0° C. and 8° C., an appropriate temperature range for preservation of follicular grafts dissected during a follicular dissection procedure. Feeder line 113, inlet 111, fluid outlet 112 and reservoirs 114 may be obtained by a variety of methods, such as, for instance, machining or molding. However, these are merely examples of methods of obtaining inlets, outlets, feeder lines and/or reservoirs and claimed subject matter is not limited in this regard.

FIG. 2 illustrates a particular embodiment of a thermal transfer circuit 200 for three TBAs 230, 231 and 232 connected in series, or in parallel, or combinations of serial and parallel connections, in one or more alternative embodiments. In a particular embodiment, TBAs 230, 231 and 232 may be configured as described in FIG. 1 and connected in series via TBA interconnection lines 201 and 202. According to a particular embodiment, input connectors 203, 204 and 205 may couple inlets 211 to an output line 207 from a pump 206 via hose couplings (not shown). Alternatively, interconnection lines 201 or 202 may be coupled to inlets 211 via hose couplings (not shown). Pump 206 may inject a fluid and/or gas media from reservoir 208 into an inlet 211 through output line 207. As described with reference to FIG. 1, such fluid and/or gas may be capable of transferring heat to or from insert pockets and/or containers (as depicted in FIG. 1) of TBAs 230, 231 and 232. According to a particular embodiment, output connectors 209, 210, and 213 may connect outlet 212 to a return line 214 flowing to reservoir 208 or to interconnection lines 201 or 202 via hose couplings (not shown). Return line 214 may return fluid and/or gas to reservoir 208 where it may be brought back to a desired temperature. Temperature of a fluid and/or gas may be controlled by a variety of methods, such as, by cooling with dry ice, or gel packs, cooling with refrigeration, cooling by compressing and releasing gas and/or heating with heating elements or chemical gel packs. Reservoir 208 may be capable of directly controlling the temperature of a fluid and/or gas and may be equipped with a control circuit (not shown) that may enable regulation of the fluid and/or gas to a particular temperature. Alternatively, reservoir 208 may be a container set in a temperature controlling device such as, for instance, a chiller (not shown). A chiller may be a refrigerated cooling system such as the “Chiller” available from VWR ™. A chiller may contain reservoir 208, the pump 206 and a control circuit (not shown) to regulate the temperature of fluid and/or gas. However these are merely examples of methods of heating or cooling fluid and/or gas and claimed subject matter is not limited in these respects.

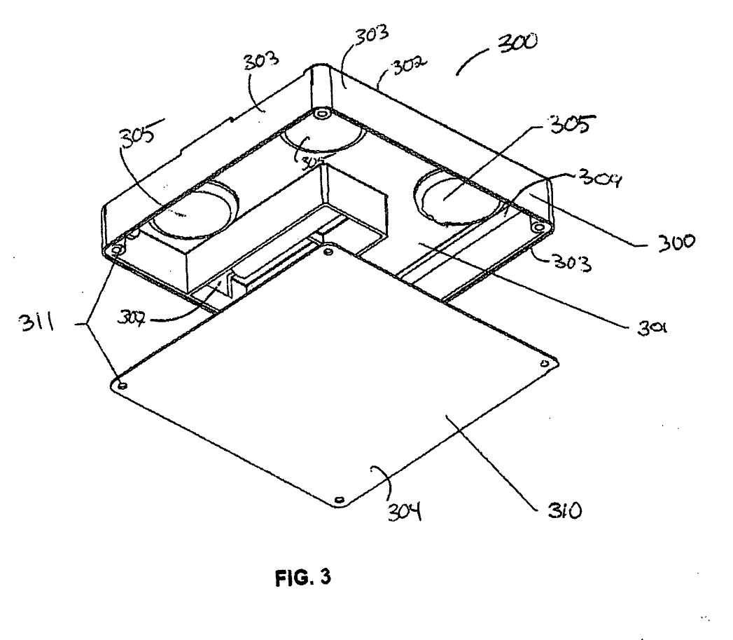

FIG. 3 illustrates an underside perspective view of a particular embodiment of a Tissue Board Assembly 300 comprising a chamber 301 for storing temperature controlling members (not shown). In a particular embodiment, a TBA 300 may comprise a top surface 302, lateral surfaces 303, a bottom surface 304 and insert pockets 305, as described in FIG. 1. TBA 300 may also comprise a light access region 307 for accessing light, as described with reference to FIG. 1. A TBA 300 may comprise one or more tool rests 309 also described with reference to FIG. 1. According to a particular embodiment, a TBA 300 may comprise a chamber 301. Such chamber 301 may be useful for enclosing temperature controlling members in close proximity to the backsides of insert pockets 305. Such temperature controlling members may comprise a variety of temperature controlling objects or devices, such as, for instance, small frozen gel packs for cooling, small chemical warming packs for heating, heat sinks for removing heat and/or heat pipes for removing heat. However, these are merely examples of temperature controlling objects or devices that may be used to control the temperature of the backsides of insert pockets and claimed subject matter is not limited in this regard.

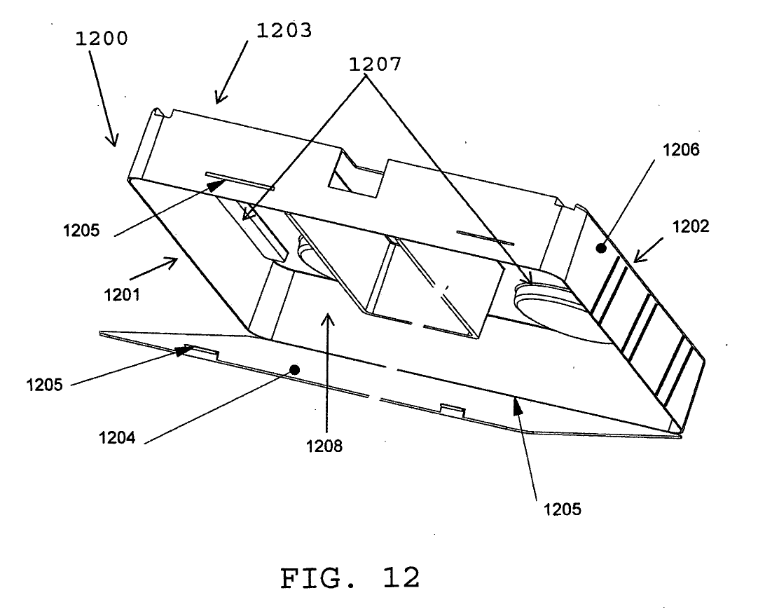

In a particular embodiment a TBA 300 may also comprise a back cover 310. Back cover 310 may comprise screw holes for removing and replacing back cover 310 using screws (not shown) or snap-in features (not shown). In a particular embodiment, back cover 310 may be removed allowing a user easy access to chamber 301 such that a user may place temperature controlling members within chamber 310 and then replace back cover 310. When back cover 310 is replaced and temperature controlling members are in place, temperature controlling members may be held securely within chamber 301 until back cover 310 is removed. In a particular embodiment, back cover 310 may be made of insulation material or covered or coated with an insulating material. Such insulation material may enable maintaining the temperature of an enclosed media within chamber 301. In a particular embodiment, a TBA 300 and back cover 310 may be made of a thin polymeric sheet material such as polycarbonate, polyester, polypropylene, polyethylene and/or ABS by thermoforming or molding processes. In a particular embodiment, wall thickness may be between 0.010 to 0.12 inches. According to a particular embodiment, TBA 300 and back cover 310 may be coupled by a hinge (as shown in FIG. 12). Back-cover 310 may be closed by snap-in features (not shown). This assembly may be made disposable or reusable depending on the material and wall thickness chosen. However, this is merely an example of a method of providing access to a chamber in a TBA and claimed subject matter is not limited in this regard.

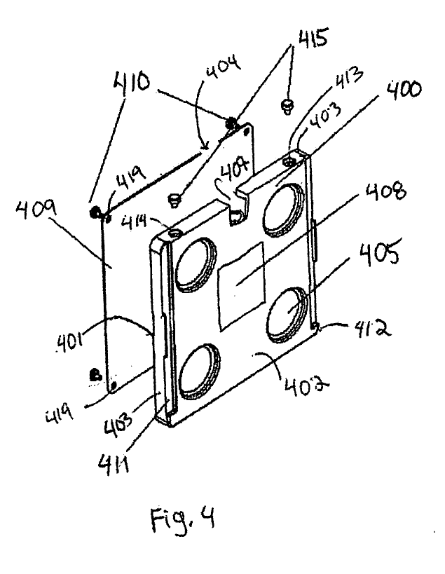

FIG. 4 illustrates a top side view of a particular embodiment of a Tissue Board Assembly 400 comprising a fluid and/or gas filled temperature control cavity 401. In a particular embodiment, a TBA 400 may comprise a main board 412 comprising a top surface 402, lateral surfaces 403 and insert pockets 405, as described in FIG. 1. TBA 400 may also comprise a cover plate 409 having a bottom surface 404 and screw holes 419, as described in FIG. 3. According to a particular embodiment, TBA 400 may further comprise a light access region 407 for accessing light, as described with reference to FIG. 1. Additionally, TBA 400 may comprise one or more tool rests 411, and a work area 408 also described with reference to FIG. 1. Rubber feet 410 may prevent TBA 400 from sliding during use. However, this is merely an example of a method of assembling a TBA and claimed subject matter is not limited in this regard.

Referring still to FIG. 4, according to a particular embodiment, a TBA 400 may comprise a cavity 401 that may be coupled to a cover plate 409 to provide a water tight seal. In a particular embodiment, cover plate 409 may be assembled to main board 412 with a water tight joint made by coupling cover plate 409 and main board 412 using screws 410. A gasket (not shown) may be disposed between cover plate 409 and main board 412. According to a particular embodiment, a variety of methods may be used to provide a water tight seal between cover plate 409 and main board 412, such as, ultrasonic welding, gluing, and/or threading to compress a seal ring or gasket between cover plate 409 and main board 412. In another embodiment, TBA 400 may comprise a cavity 401 made by molding, thermoforming, machining and/or casting. Accordingly, it may not be necessary to place a cover plate on the backside of main board 412. Cover plate 409 may be positioned anywhere on main board 412 such that it creates a cavity (not shown) with main board 412. Alternatively, cover plate 409 may be eliminated where certain molding and/or machining processes are used to manufacture TBA 400. However, these are merely examples of methods of providing a water tight seal between a cover plate and a main board and placement of a cover plate and claimed subject matter is not limited in this regard.

In a particular embodiment, cavity 401 may be filled with a fluid and/or a gas. According to a particular embodiment, the fluid and/or gas may be in contact with the backside (not shown) of insert pockets 405. Such fluid and/or gas may be brought to a particular temperature and may be capable of enabling temperature control of insert pockets 405. In a particular embodiment, sealed cavity 401 may be filled by injecting fluid and/or gas into inlet hole 413. After filling, inlet hole 413 and outlet hole 414 may be plugged with plugs 415. According to a particular embodiment, fluid may be injected to a prescribed mark (not shown) then inlet hole 413 and outlet hole 414 may be closed with plugs 415. Such plugs 415 may provide a water tight seal to inlet 413 and outlet 414 by a variety of methods such as but not limited to threading, gluing, and/or force fitting. Additionally plugs 415 may be made of a variety of materials, such as but not limited to vulcanized rubber and/or cork. According to a particular embodiment, TBA 400 may be exposed to a specific temperature environment such as a freezer or warm water bath in order to bring the fluid and/or gas within cavity 401 to a desired temperature. Alternatively, fluid and/or gas may be circulated into and out of cavity 401 through inlet hole 413 and outlet hole 414. Accordingly, fluid and/or gas may be temperature controlled outside of cavity 401. However, these are merely examples of methods of providing temperature controlled fluid and/or gas within a cavity of a TBA and claimed subject matter is not so limited.

In a particular embodiment, insert pockets 405 and containers (not shown) as described with reference to FIG. 1 may comprise a TBA 400. According to a particular embodiment, insert pockets 405 and/or containers (not shown) as discussed with reference to FIG. 1 may be comprised of material with high thermal conductivity, such as, for instance, copper, aluminum, and/or glass. In a particular embodiment, main board 412 and cover plate 409 may be comprised of insulating materials having a low thermal conductivity such as plastic, polystyrene and/or fiberglass. However, these are merely examples of kinds of materials that may be used to make containers and insert pockets and claimed subject matter is not limited in this regard.

FIG. 5 illustrates a particular embodiment of a Tissue Board Assembly. In a particular embodiment, a TBA 500 may comprise a main board 512 comprising a top surface 502, lateral surfaces 503, a bottom surface 504 and work area 508 as described with reference to FIG. 1. In a particular embodiment, TBA 500 may comprise inlet 511, outlet 512 and feeder line 513. According to a particular embodiment, feeder line 513 may be coupled to inlet 511 and outlet 512, as described with reference to FIG. 1. Additionally, TBA 500 may have at least one rubber foot 510 to help prevent sliding of TBA 500 during use, as described with reference to FIG. 1. However, this is merely an example of a method of assembling a TBA and claimed subject matter is not limited in this regard.

In a particular embodiment, TBA 500 may comprise at least one insert pocket 505. Such a pocket may be an insert or an integral part of the main board 512. Such insert pockets may have a variety of shapes and dimensions, such as, for instance, being substantially circular with a diameter of about 1.5 inches and a depth of about 0.25 inches. Insert pockets 505 may be the containers in FIG. 5 which may contain liquid media suitable for keeping tissue grafts hydrated. Alternatively, insert pockets 505 may contain such liquid media without the use of containers. According to a particular embodiment, insert pockets 505 may be comprised of materials having high thermal conductivity, such as was described with reference to FIG. 4. Insert pockets 505 may be in thermal contact with inlet 511, outlet 512 and/or feeder line 513. According to a particular embodiment, inlet 511, feeder line 513 and outlet 512 may circulate a fluid and/or gas capable of transferring heat to or from insert pockets 505. However, these are merely examples of insert pockets and methods of exchanging heat between a fluid and/or gas and insert pocket and containers and claimed subject matter is not limited in this regard.

Referring still to FIG. 5, in a particular embodiment, TBA 500 may comprise at least one slot 514. In a particular embodiment, TBA 500 may have two such slots 514. Such slots may have a variety of shapes and dimension such as, for instance, being about 0.5 inches wide and 1.87 inches long and having a depth of 0.25 inches. According to a particular embodiment, pads 515 may be disposed within slots 514. During a strip hair transplant procedure, a user may use a needle to pin tissue grafts to pads 515 in order to prepare tissues for transplant by stripping, stretching, and measuring. Such pads 515 may be comprised of a variety of materials, such as, for instance, rubber and/or cork. However, these are merely examples of materials that may comprise pads and claimed subject matter is not limited in this regard.

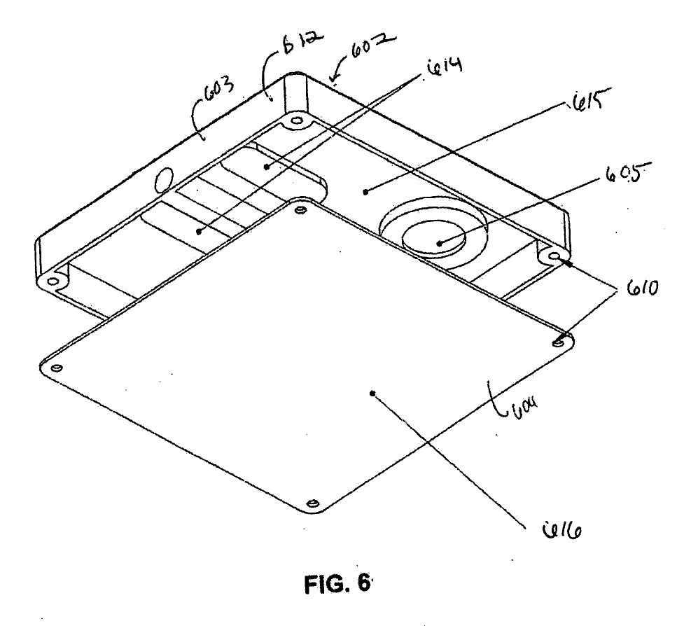

FIG. 6 illustrates a particular embodiment of a Tissue Board Assembly capable of facilitating tissue graft dissection comprising a temperature control cavity 615. In a particular embodiment, a TBA 600 may comprise various features discussed above. For instance, TBA 600 may comprise a main board 612 comprising a top surface 602, lateral surfaces 603, a bottom surface 604 and work area (not shown) as described with reference to FIG. 4. Additionally, in a particular embodiment, TBA 600 may comprise insert pocket 605, rubber feet holes 610 for at least one rubber foot 610 as described with reference to FIG. 4. However, these are merely examples of previously described components that a TBA may comprise and claimed subject matter is not limited in this regard.

According to a particular embodiment, insert pockets 605 may be in thermal contact with substances held within temperature control cavity 615. Cavity 615 may contain a free flowing fluid and/or gas. Alternatively, temperature control cavity 615 may contain one or more cooling gel packs, dry ice, chemical warming packs, heat sinks and/or heat pipes, as described with reference to FIGS. 3 and 4. Such a temperature control cavity 615 may be sealed with cover 616, as described with reference to FIG. 4. Alternatively, main board 612 may comprise an inlet and an outlet (not shown). Such inlet and outlet may allow fluid and/or gas to flow into and out of cavity 615.

In a particular embodiment, TBA 600 may comprise at least one slot 614. According to a particular embodiment, pads (not shown) may be disposed within slots 614. As discussed with reference to FIG. 5, during a strip hair transplant procedure, a user may use a needle to pin tissue grafts to pads in order to prepare tissues by stripping, stretching, and/or measuring. Such pads may be comprised of a variety of materials, such as rubber and/or cork. However, this is merely an example of a method of configuring various features of a TBA and claimed subject matter is not limited in this regard.

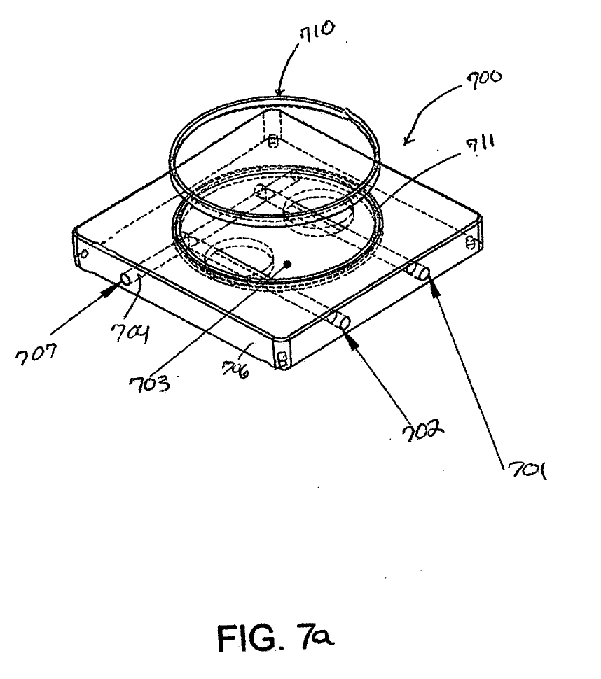

FIG. 7a and FIG. 7b illustrate different embodiments of a Tissue Board Assembly comprising a shallow dish.

FIG. 7a illustrates a particular embodiment of a Tissue Board Assembly 700 comprising a shallow dish 710. In a particular embodiment, a TBA 700 may comprise a container capable of being inserted into pocket insert 711. Such a container may be, for instance, a shallow dish 710. Such a shallow dish may be comprised of a variety of materials such as, for instance, stainless steel, plastic and/or Pyrex®. During a follicular dissection procedure, a shallow dish 710 may be used to contain a solution suitable for preservation of tissue grafts. Tissue grafts may be transferred to shallow dish 710 after harvesting from a patient during a follicular dissection. However, this is merely an example of a use of a TBA comprising a shallow dish and claimed subject matter is not limited in this regard. As described with reference to particular embodiments in FIG. 1 and FIG. 5, a shallow dish 710 may be in thermal contact with inlet 701 and outlet 702 and may fit within pocket insert 703. Feeder line 704 may extend across inlet 701 and outlet 702 to lateral surface 706 and may be sealed with a plug 707. Such a plug 707 may be made from a variety of materials, such as, for instance, plastic, rubber and/or metal. However, this is merely an example of materials a plug may comprise and claimed subject matter is not limited in this regard.

FIG. 7b illustrates an underside view of a particular embodiment of a Tissue Board Assembly 700 comprising a shallow dish 710. In a particular embodiment, a TBA may comprise a temperature control cavity 717. Cavity 717, as described with reference to FIGS. 3, 4 and 6 may be capable of containing substances useful to control the temperature of insert pocket 718 and any container held within insert pocket 718. In a particular embodiment, such a container may be a shallow dish 710, as described with reference to FIG. 7a According to a particular embodiment, cavity 717 may contain a free flowing fluid and/or gas. Alternatively, temperature control cavity 717 may contain temperature controlling objects or devices, such as, for instance, one or more cooling gel packs, dry ice, chemical warming packs, heat sinks and/or heat pipes. However, this is merely an example of temperature controlling objects or devices and claimed subject matter is not limited in this respect.

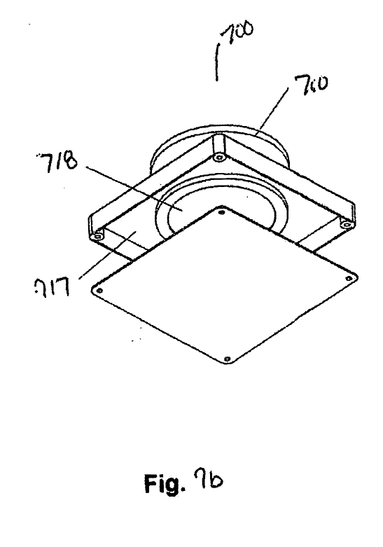

FIG. 8 illustrates a particular embodiment of a Tissue Board Assembly 800 comprising recessed work area 801 and a mesh screen 802. In a particular embodiment, a TBA 800 may comprise a recessed work area 801. Such a recessed work area 801 may disposed on top surface 820 of main board 812, may have a smooth or textured surface and may comprise a variety of dimensions. For instance, recessed work area 801 may be substantially rectangular and may have a recess. Such a recess may be about 0.01 inches to 0.1 inch in depth. Additionally, the surface may comprise a variety of materials such as, for instance, rubber and/or plastic. Such a recessed work area 801 may allow a user to work on tissue grafts extracted during a follicular dissection procedure in a thin film of solution for keeping tissue grafts hydrated while being prepared. However, these are merely examples of dimensions and materials that may comprise a recessed work area and claimed subject matter is not limited in this regard. Additionally, TBA 800 may comprise a mesh screen 802 disposed over an insert pocket 803. According to a particular embodiment, a user may be able to keep tissue hydrated in insert pocket 803 after preparing grafts in recessed work area 801 over mesh screen 802. Mesh screen 802 may be submerged in the solution in insert pocket 803. In a particular embodiment, the solution may remain in insert pocket 803 in main board 812. A handle 804 may be used to remove the mesh screen 802. Additionally, according to a particular embodiment, a TAB 800 may comprise one or more containers 813 held within one or more recesses 814 in main board 812. In a particular embodiment, containers 813 and corresponding recesses 814 may comprise a variety of shapes such as circular, oval, rectangular, and/or contoured. Such containers may be useful for holding and preserving tissue grafts harvested during a follicular dissection procedure and to transfer grafts ready for transplant from main board 812 to the recipient area. In a particular embodiment, TBA 800 may have a variety of other features, such as, rubber pads 815 and a light access region 816 as described above with reference to FIG. 1 and FIG. 5. However, this is merely an example of a TBA having a variety of features introduced in this embodiment and/or described in other embodiments discussed above and claimed subject matter is not limited in this regard.

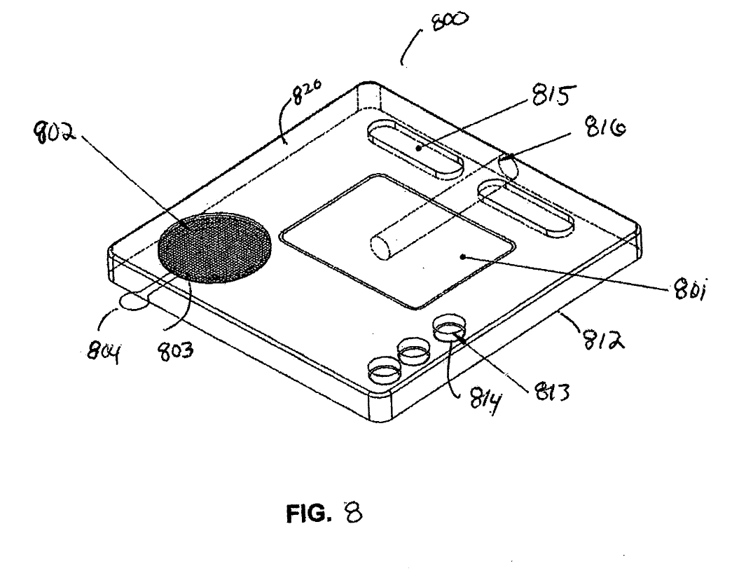

FIG. 9 illustrates a particular embodiment of TBA 900 comprising multiple surface features including removable insert 903, round container 906 rectangular container 905 and back insert plate 916. In a particular embodiment, a TBA 900 may comprise a top surface 910, lateral surfaces 911, a bottom surface (not shown) and insert pockets 901, as described above with reference to FIG. 1. In a particular embodiment, TBA 900 may comprise additional features already discussed in detail above such as inlet 913 and outlet 914 and light access region 907. However, these features are merely examples of a variety of features a TBA may comprise and claimed subject matter is not limited in these regards.

In a particular embodiment, TBA 900 may comprise removable insert 903 comprising a work area 917. In a particular embodiment, removable insert 903 may be disposed over a lighting recess 902. Such insert 903 may experience a degree of wear and tear on work areas 917 and it may be useful from time to time to be able to replace insert 903. In a particular embodiment, work area 917 on removable insert 903 may be textured. However, this is merely an example of a removable insert for a TBA and claimed subject matter is not limited in this regard.

During a follicular dissection procedure, grafts harvested from various tissues may be various colors. Hence, insert 903 comprising various colors may facilitate preparation of tissue grafts by allowing a surgeon/user to see tissue more clearly against a contrasting background color. Thus, removable insert 903 may comprise a variety of materials and may also comprise a variety of colors. However, this is merely an example of uses of removable inserts comprising various materials and various colors and claimed subject matter is not limited in this regard.

In a particular embodiment, removable insert 903 may be disposed over a lighting recess 902 and comprise a transparent material. Such transparent materials may allow light to filter though and to directly illuminate a tissue specimen on work area 917. In a follicular dissection procedure, using different color transparencies based on the color of the hair in the follicular graft may facilitate graft preparation by allowing a surgeon to see the hair and follicle more clearly. The main board 912 may be clear or opaque and may be made from a variety of materials as discussed above. However, these are merely examples of the variety of materials that may comprise removable insert 903 and claimed subject matter is not limited in this regard.

In a particular embodiment, a back insert plate 916 may be disposed underneath removable insert 903 on a bottom surface of TBA 900. In a particular embodiment, back insert plate 916 may be capable of reflecting light onto work area 917 of removable insert 903 and/or may comprise various colors and reflective properties. According to a particular embodiment, back insert plate 916 may provide a background color appropriate to a particular hair and/or tissue color to facilitate visualization of tissue grafts by a user/surgeon during a follicular dissection procedure, for instance. According to a particular embodiment, back insert plate 916 may comprise a variety of materials, such as, for instance, metals, tinted plastics and/or tinted glass. However, this is merely an example of the variety of materials that may comprise a removable insert and claimed subject matter is not limited in this regard.

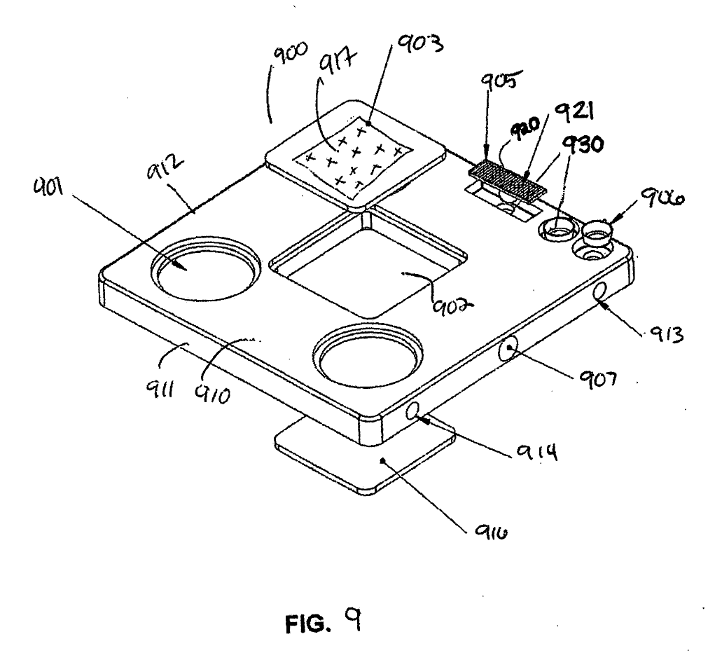

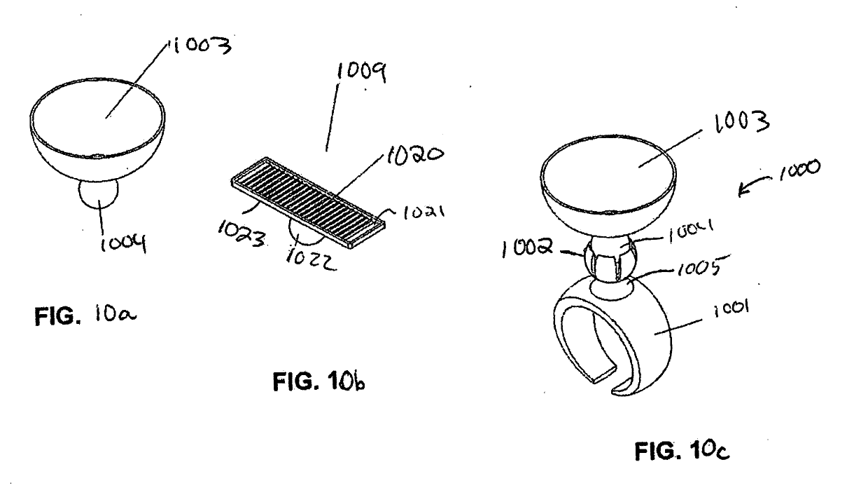

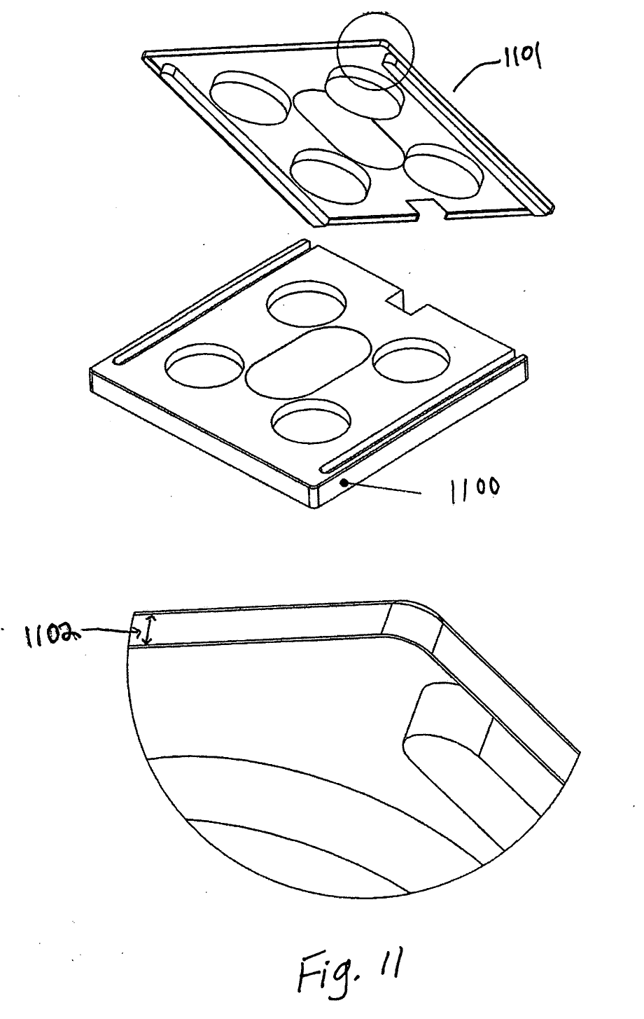

Referring still to FIG. 9, a TBA 900 may comprise one or more round containers 906 and/or one or more rectangular containers 905. In a particular embodiment, containers 905 and 906 may be disposed on a top surface 910 of a main board 912 of a TBA 900. TBA 900 may comprise container recesses 930 which may hold containers 905 and 906. According to a particular embodiment, containers 905 and 906 may be a variety of shapes, such as, oval, rectangular and/or circular. In a particular embodiment, containers 905 and 906 may be useful for transferring and/or preserving grafts during a follicular dissection procedure. Accordingly, rectangular containers 905 may comprise top 920. Top 920 may have several grooves 921. Such grooves 921 may enable organization of tissue grafts by placement of tissue grafts along grooves 921. Although not shown in FIG. 9, round containers 906 may also have tops comprising grooves arranged in a circular pattern around the circumference. Rectangular container 905 may be capable of holding a thin film of solution within grooves 921. In a particular embodiment, rectangular containers 905 and 906 may be removable and may be assembled to a finger mounted container support 1001 (as shown in FIGS. 10a through 10c).

Referring now to FIG. 10, FIG. 10b, and FIG. 10c, a container fastening assembly 1000 is illustrated. In a particular embodiment, container support 1005 may comprise a fastening member 1001 for attachment to the finger of the user/surgeon. Fastening member 1001 may comprise a variety of objects capable of fastening, such as, for instance, a ring, a clip, a magnet, and/or a Velcro® strip. Alternatively, fastening member 1001 may be applied to the wrist or hand of a user to free the fingers. However, these are merely examples of methods of fastening a container fastening assembly and claimed subject matter is not limited in this regard.

FIG. 10a, illustrates a particular embodiment of a round container 1003. In a particular embodiment, container 1003 may comprise a ball 1004 disposed on the bottom portion of container 1003. Returning to FIG. 10c, container 1003 may reside within a socket 1002. Alternatively, container fastening assembly 1000 may have a ball disposed on a top portion of fastening member 1001 and a socket may be disposed on the bottom portion of container 1003. In a particular embodiment, container fastening assembly 1000 may facilitate organization of tissue grafts. Such container fastening assembly 1000 may also facilitate the transfer of follicular grafts to a patient recipient area during a follicular transplant procedure by allowing a user to wear container fastening assembly 1000 and have it close at hand while transplanting follicular grafts into the recipient area during a follicular transplantation procedure. Alternatively, container fastening assembly 1000 may be fastened close to the user/surgeon's working area such as on the table a patient is lying on during a procedure or fastened to the patient for easy accessibility. However, these are merely examples of methods of fastening a container fastening assembly and claimed subject matter is not limited in this regard.

FIG. 10b illustrates an embodiment of a rectangular container 1009. Top 1020 may have several grooves 1021. Such grooves 1021 may extend across the width of rectangular container 1009. Profiles of grooves 1021 may enable organization of tissue grafts by placement of tissue grafts along grooves 1021. However, this is merely an example of grooves that may be disposed on a container and claimed subject matter is not limited in this regard.

In a particular embodiment, a connector 1022 may reside on the bottom portion 1023 of container 1009. Such a connector may comprise a variety of objects capable of facilitating a connection between at least two objects, such as, for instance a ball, socket, clip snap, hook and/or button. In a particular embodiment, a connector 1022 may comprise a ball. Such a ball may enable attachment of rectangular container 1009 to a fastening member having a socket (such as socket 1002 shown in FIG. 10c).

According to a particular embodiment, socket 1002 in FIG. 10c may facilitate assembly and disassembly of the container fastening assembly 1000. Additionally, container fastening assembly 1000 may allow rotation of container 1003. Such rotation may facilitate graft placement. When the container wearing finger/hand is held in a certain position, the graft container 1003 may be rotated in space to render itself closer to the specific incision site and more accessible to the hand a user is working with. However these are merely examples of ways in which to assemble a container fastening assembly and claimed subject matter is not so limited.

FIG. 11 illustrates a Disposable Tissue Board 1101 disposed over a Tissue Board Assembly 1100. In a particular embodiment, a Disposable Tissue Board 1101 may be made by a variety of processes, such as, for instance, molding, thermoforming, and vulcanizing, and/or similar processes. Additionally, Disposable Tissue Board 1101 may be made from a variety of materials such as plastic and/or rubber, According to a particular embodiment, Disposable Tissue Board 1101 may comprise a shelled structure with a wall thickness of about 0.007 inches to 0.12 inches. However, these are merely examples of materials and methods used to produce a Disposable Tissue Board and claimed subject matter is not limited in this regard.

FIG. 12 illustrates an embodiment of a Disposable Tissue Board (DTB) 1200 capable of use as a stand-alone device. In a particular embodiment, DTB 1200 may be utilized alone or as a cover for a TBA (not shown). According to a particular embodiment, DTB 1200 may be utilized alone. Such a stand alone DTB 1200 may comprise a wall 1201 having a thickness, for instance, of 0.012 inches to 0.120 inches. In a particular embodiment, various support structures incorporated into wall 1201 may allow DBT 1200 to resist certain forces, such as, those caused by the user preparing a specimen on the surface 1203 of DTB 1200. Such support structures may comprise ribbing, thin strips of metal, plastic attachments, and/or plastic or metal inserts assembled or joined either to the main board 1206 or the cover plate 1204. In a particular embodiment, such support structures may comprise ribs 1202, for instance. Such ribs 1202 may, additionally, enable DTB 1200 to support various features 1207 described above with reference to a TBA. Such features may comprise insert pockets, removable inserts, containers, tool rests, pads, mesh screens and/or work areas. According to a particular embodiment, grooves and other surface features of components for a TBA (as described above) may be reproduced on surface 1203 of DTB 1200. However, these are merely examples of various features of a DTB and claimed subject matter is not so limited.

In a particular embodiment, DTB 1200 may have a bottom cover 1204. Such a bottom cover 1204 may have fasteners 1205 on lateral sides of bottom cover 1204. Such fasteners may include snap-on features, hinges and/or other fasteners, for instance. Fasteners 1205 may secure bottom cover 1204 to main board 1206. In a particular embodiment, bottom cover 1204 may enable the use of thermal control substances or objects within cavity 1208, such as, for instance, hot gel packs or cold gel packs (not shown) to be enclosed in the cavity 1208 between the main board 1206 and the bottom cover 1204. However, this is merely an example of a configuration of a DTB and claimed subject matter is not limited in this regard.

While certain features of claimed subject matter have been illustrated as described herein, many modifications, substitutions, changes and equivalents will now occur to those skilled in the art. It is, therefore, to be understood that the appended claims are intended to cover all such embodiments and changes as fall within the true spirit of claimed subject matter.

What is claimed is:

1. A tissue board assembly comprising:
a main board comprising a surface, the surface comprising top side, a bottom side and one or more lateral sides;
at least one insert pocket disposed in a recess on the top side of the main board;
the main board having and interior region, the interior region being capable of containing a substance in thermal contact with at least one insert pocket, the interior region comprising;
an inlet opening to the surface of the main board to allow the substance to enter the interior region; and
an outlet opening to the surface of the main board to allow the substance to exit the interior region.

2. The tissue board assembly of claim 1 and further comprising at least one reservoir disposed under the at least one insert pocket, the reservoir intersecting with the interior region and being capable of receiving a substance flowing through the interior region.

3. The tissue board assembly of claim 1 and further comprising one or more of the following: at least one container, at least one work area or at least one removable insert, or combinations thereof.

4. The tissue board assembly of claim 1 and further comprising at least one tool rest disposed on one or more of the top side, the bottom side, or the lateral sides, or combinations thereof.

5. The tissue board assembly of claim 1, further comprising a light access region extending into the interior region of the main board.

6. The tissue board assembly of claim 1, further comprising at least one foot disposed on a bottom side of the main board.

7. The tissue board assembly of claim 1 wherein the inlet opening is coupled to a conduit capable of passing a substance into the interior region and wherein the outlet opening is coupled to a conduit capable of carrying a substance away from the interior region.

8. The tissue board assembly of claim 1, further comprising at least one pad disposed within a recess on the top side of the main board.

9. The tissue board assembly of claim 1, further comprising at least one mesh screen disposed within at least one insert pocket on the top side of the main board.

10. The apparatus of claim 1, further comprising an insert, the insert comprising a top side, a first set of lateral walls and a bottom side, the top side of the insert comprising a work area and wherein the recess in the top side of the main board comprises a second set of lateral walls, the second set of lateral walls being taller than the first set of lateral walls such that a fluid may be retained at least temporarily over the work area of the top side of the insert when the insert is disposed within the recess.

11. A tissue board assembly comprising:
a main board comprising a top side and one or more lateral sides;
at least one insert pocket disposed in a recess on the top side of the main board;
the at least one insert pocket comprising an inner surface and a backside surface; and
at least one thermal control cavity disposed within the main board, the cavity comprising an inside surface, the inside surface comprising at least one backside surface of the at least one insert pocket.

12. The tissue board assembly of claim 11 and further comprising a cover plate capable of being coupled to the main board.

13. The tissue board assembly of claim 11 and further comprising one or more of the following on the surface of the main board: at least one container, at least one work area or at least one removable insert, or combinations thereof.

14. The tissue board assembly of claim 11 and further comprising at least one tool rest disposed on of the main board.

15. The tissue board assembly of claim 11 and further comprising a light access cavity comprising an opening to a lateral side of the main board.

16. The tissue board assembly of claim 12 and further comprising at least one foot disposed on a surface of the cover plate.

17. The tissue board assembly of claim 11 and further comprising at least one access hole disposed on the surface of the main board, the access hole being capable of allowing passage of a substance into or out of the thermal control cavity.

18. The tissue board assembly of claim 11 and further comprising at least one mesh screen disposed over at least one insert pocket on the top side of the main board.

19. The apparatus of claim 11, further comprising an insert, the insert comprising a top side, a first set of lateral walls and a bottom side, the top side of the insert comprising a work area and wherein the recess in the top side of the main board comprises a second set of lateral walls, the second set of lateral walls being taller than the first set of lateral walls such that a fluid may be retained at least temporarily over the work area of the top side of the insert when the insert is disposed within the recess.

20. The tissue board assembly of claim 11 and further comprising at least one pad disposed within a recess on the top side of the main board.

21. A tissue board assembly comprising:
a main board comprising a surface, the surface comprising a top side, a bottom side and one or more lateral sides; and
at least one removable insert comprising a work area disposed on the top side of the main board.

22. The tissue board assembly of claim 21 and further comprising;
at least one insert pocket disposed in a recess on the top side of the main board, the insert pocket comprising a backside surface;
the main board having an interior region, the interior region being capable of containing a substance in thermal contact with at least one insert pocket, the interior region comprising;
an inlet opening to the surface of the main board to allow the substance to enter the interior region; and
an outlet opening to the surface of the main board to allow the substance to exit the interior region.

23. The tissue board assembly of claim 22 and further comprising at least one reservoir disposed under the at least one insert pocket, the reservoir intersecting with the interior region and being capable of receiving a substance flowing through the interior region.

24. The tissue board assembly of claim 22 and further comprising one or more of the following disposed on the top side of the main board; at least one container, at least one work area or at least one removable insert, or combinations thereof.

25. The tissue board assembly of claim 22 and further comprising at least one tool rest disposed on the surface of the main board.

26. The tissue board assembly of claim 22 and further comprising a light access region disposed within the main board.

27. The tissue board assembly of claim 22 and further comprising at least one foot disposed on the bottom surface of the main board.

28. The tissue board assembly of claim 22 wherein the inlet opening is coupled to a conduit capable of passing a substance into the interior region and wherein the outlet opening is coupled to a conduit capable of carrying a substance away from the interior region.

29. The tissue board assembly of claim 22 and further comprising at least one pad disposed within a recess on the top side of the main board.

30. The tissue board assembly of claim 22 and further comprising at least one mesh screen disposed over the at least one insert pocket on the top side of the main board.

31. The apparatus of claim 22, further comprising an insert, the insert comprising a top side, a first set of lateral walls and a bottom side, the top side of the insert comprising a work area and wherein the recess in the top side of the main board comprises a second set of lateral walls, the second set of lateral walls being taller than the first set of lateral walls such that a fluid may be retained at least temporarily over the work area of the top side of the insert when the insert is disposed within the recess.

32. A tissue board assembly comprising:
a first main board comprising a surface, a top side, a bottom side and one or more lateral sides; and
at least one removable insert comprising a work area disposed on the top side of the first main board.

33. The tissue board assembly of claim 32 and further comprising;
at least one insert pocket disposed in a recess on the top side of the first main board, the at least one insert pocket comprising a backside surface; and
at least one thermal control cavity disposed within the first main board, the cavity comprising an inside surface, the inside surface comprising at least one backside surface of the at least one insert pocket.

34. The tissue board assembly of claim 33 and further comprising a cover plate capable of being coupled to the first main board.

35. The tissue board assembly of claim 33 and further comprising one or more of the following disposed on a top side of the first main board; at least one container, at least one pad or at least one tool rest or combinations thereof.

36. The tissue board assembly of claim 33 and further comprising a light access region comprising an opening to a lateral side of the first main board.

37. The tissue board assembly of claim 34 and further comprising at least one foot disposed on a surface of the cover plate.

38. The tissue board assembly of claim 33 and further comprising at least one access hole disposed on the surface of the first main board, the access hole being capable of allowing passage of a substance into and/or out of the thermal control cavity.

39. The tissue board assembly of claim 33 and further comprising at least one mesh screen disposed over the at least one insert pocket on the top side of the first main board.

40. The apparatus of claim 33, further comprising an insert, the insert comprising a top side, a first set of lateral walls and a bottom side, the top side of the insert comprising a work area and wherein the recess in the top side of the main board comprises a second set of lateral walls, the second set of lateral walls being taller than the first set of lateral walls such that a fluid may be retained at least temporarily over the work area of the top side of the insert when the insert is disposed within the recess.

41. The tissue board assembly of claim 33 and further comprising at least one pad disposed within a recess on the top side of the first main board.

42. The tissue board assembly of claim 33 and further comprising a back insert plate disposed on the bottom side of the first main board.

43. The tissue board assembly of claim 33 further comprising;
the first main board further comprising surface features;
a disposable tissue board comprising;
a second main board comprising a surface, said surface comprising a top side, and one or more lateral sides;
a cavity disposed on the underside of the second main board, the cavity comprising a negative impression of surface features of the first main board.

44. A tissue board assembly comprising:
a main board;
at least one insert pocket disposed within the main board, the insert pocket comprising a first connector; and
at least one removable container capable of being disposed over the first connector, the removable container comprising a bottom portion wherein the bottom portion comprises a second connector.

45. The tissue board assembly of claim 44 wherein the first connector is a ball and the second connector is a socket.

46. The tissue board assembly of claim 44 wherein the first connector is a socket and the second connector is a ball.

47. A container fastening assembly comprising:
a container comprising a recess and a first connector;
a fastening member comprising a second connector; and
the first connector and the second connector capable of coupling the container to the fastening member.

48. The container of claim 47 wherein the first connector is a ball and wherein the second connector is a socket.

49. The container of claim 47 wherein the first connector is a socket and wherein the second connector is a ball.

50. The container of claim 47 wherein the fastening member is one or more of the following alone or in combination; a ring, a magnet, a clip or a Velcro® strip.

51. A disposable tissue board assembly comprising:
a main board comprising a top side, at least one lateral side and a cavity; and
at least one surface feature disposed within the top side of the main board.

52. The disposable tissue board assembly of claim 51, further comprising;
a bottom cover capable of being coupled to the main board at least temporarily; and
fastening members capable of coupling the bottom board to the main board, at least temporarily.

53. The disposable tissue board assembly of claim 52 wherein the at least one lateral side wall comprises at least one support structure.

54. An apparatus comprising:
means for retaining a specimen taken during a surgical procedure; and
means for controlling the temperature of the specimen.

55. The apparatus of claim 54 wherein the means for controlling the temperature further comprises a means for circulating fluid or gas through an interior region of the apparatus.

56. The apparatus of claim 54 wherein the means for controlling the temperature further comprises a means for filling a cavity within an interior region with a substance.

57. The apparatus of claim 54 wherein the means for retaining a specimen further comprises:
means for collecting the specimen; and
means for means for hydrating the specimen.

58. The apparatus of claim 54 wherein the means for retaining a specimen further comprises:
means for storing the specimen; and
means for draining fluid off of the specimen.

59. The apparatus of claim 54 wherein the means for retaining a specimen further comprises:
means for preparing the specimen; and
means for securing the specimen in place.

60. The apparatus of claim 59 wherein the means for preparing the specimen further comprises means for providing a friction force sufficient to substantially prevent a specimen from sliding while being prepared.
